(19) United States
(12) Patent Application Publication
(10) Pub. No.: US 2021/0322201 A1
(43) Pub. Date: Oct. 21, 2021

(54) Postoperative Buttocks Recovery Bed

(71) Applicant: Alexandria Elizabeth Tate, East Point, GA (US)

(72) Inventor: Alexandria Elizabeth Tate, East Point, GA (US)

(21) Appl. No.: 17/365,613

(22) Filed: Jul. 1, 2021

(51) Int. Cl. A61F 5/30; A61G 7/07; A61G 7/05

(52) U.S. Cl. CPC A61F 5/30; A61G 7/0503; A61G 7/07

(57) ABSTRACT

The present invention discloses a devise constructed to prevent any pressure caused to a patient while laying and sleeping in a flat position on their backside after surgery to the buttocks area, consisting of a negative pressure site where the buttocks sleeps and rests. The device is used post-surgery during recovery following a Brazilian Butt Lift surgery including, but not limited to, Fat Transfer, Fat Grafting, Liposuction, Butt Shots, Butt Implants, or any surgical procedure to the Gluteal, Buttocks, or Coccyx area. The present device solves this current problem of a patient being uncomfortable or in an unnatural position during rest or sleep while recovering 24 hours a day for 6-8 weeks; and prevents pressure to the buttocks having direct contact with any surface during sleep and resting while inside the negative pressure site, supporting the head, neck, legs, and feet in an elevated position.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

### Field of the Invention

The present invention relates to a supportive device to the buttocks post-surgery. More particularly the invention relates to postoperative buttocks recovery cushions and buttocks postop care, a postoperative buttock recovery bed that provides total body support, while lying flat on the patient's backside no pressure to buttocks is applied.

### Description of the Related Art

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

When a patient undergoes a gluteal surgery, the patient initially wakes up with no device to protect the buttocks, back, head, neck, or feet on the hospital bed. The patient wakes up out of surgery and they are lying on their buttock with no cushion gear to support any part of the body. A patient is told if applied pressure to by sitting or lying on the augmented buttocks the patient can destroy the results of the fat grafting or implant after a BBL (Brazilian Butt Lift) or any gluteal procedure. When a patient first wakes up post anesthesia they are lying on their back in extreme pain, their body from head to toes is bruised front and backside. Some patients may undergo other procedures at the same time such as a breast augmentation. The doctors instruct patients to recover lying on their stomach or to lie on their sides rotating from time to time applying no pressure to buttocks area. The problem arises is when a patient wakes up on one's buttocks and the patient will have to position themselves on their side or front side which is not feasible nor comfortable, however this is the current solution for postoperative gluteal surgical patients. One additional problem arises if a patient has undergone breast surgery and gluteal surgery at the same time but is told by doctors to lay recovering on their front side so that no pressure is on the buttocks, this causes pressure to the breast recovering. There is a need for a device that will negatively apply pressure to the buttocks and the same time support head, neck, body, buttocks, and feet allowing a patient to recover on their backside lying in a flat position on top of a traditional bed.

Doctor Kamran Azad, Md states on Recovering from a Brazilian butt lift ASPS (plasticsurgery.org), “One of the most important recovery tips regarding Brazilian butt lift surgery is to not sit directly on your buttocks for at least eight weeks after the procedure. Patients are often advised to sleep on their stomach or sides, and if you absolutely do have to sit, it is recommended to use a donut pillow or inflatable pillow. Placing the pillow behind your legs is helpful to avoid putting pressure on the fat graft by raising the buttock. This is very important to achieve long lasting results, as sitting directly on the buttocks can affect blood circulation in the area. Reduced blood circulation could destroy the fat cells transferred to the buttocks, though it should be noted that up to 40% of the transferred fat may not survive, regardless of if a patient has or has not sat down. After the first eight weeks of recovery, it is important to still use a cushion when sitting, and to avoid sitting for prolonged periods of time. It is also advised to continue to avoid sleeping on your back for the first eight weeks after your surgery.” All doctors' recommendations are the same as Dr. Azad's, no lying on the backside to recover post gluteal surgery.

Furthermore, there is a need for a device that enables a postop patient upon waking up post anesthesia to recover protecting the results of the buttocks augmentation comfortably and on their backside while supporting all other areas of the body post gluteal surgery. Gluteal surgical enhancements are expensive, and the procedure follows strict recovery protocols. It is imperative for the patient to follow a doctor's postop recovery directions to achieve best results from one's surgery. Once such protocol is a patient is required to recover lying on their stomach for months to recover. The BBL (Brazilian Butt Lift) procedure requires a patient to have fat removed from one area of the body via liposuction and injected into the buttocks area. Some patients also have implants into the buttocks and there is currently no device to support a patient recovering on their back after any gluteal surgical procedure that supports the whole body and simultaneously removing pressure from the buttocks. Gluteal cosmetic procedures are extremely painful when recovering and there is a need for comfort and negative pressure therapy as well as shifting a patients recovery process from the front side to the backside.

Website Memory foam cushion—A piece of NASA tech in your wheelchair (invacare.eu.com) states “Okay, it's memory foam. But what is memory foam? Without being overly technical, it consists mainly of polyurethane—an organic polymer—as well as additional chemicals that increase its viscosity and density, as per manufacturer's needs or wishes. It is, therefore, often referred to as “viscoelastic” foam.

Memory foam derives its viscoelastic properties from several effects, all due to the internal structure of the material, mentioned above. Now, as any other foam or sponge, memory foam wants to restore its original structure when it is deformed. This effect is generated by the deformed porous material pushing outwards to restore its structure against an applied pressure.

However, unlike other foams, in memory foam there are three opposite effects which work against this inherent need to restore its original structure. These, combined, work to slow the regeneration of the original structure of the foam, which allows for particularly useful applications, like memory foam cushion. Another useful property of the material is that higher-density memory foam softens in reaction to body heat, allowing it to mold to a warm body in a few minutes. It is also breathable, and newer generations of memory foam contain phase-change gel which can produce temperature stabilization or cooling effect, or even provide aromatherapy.

Yes, gel. The fact that gel in your wheelchair cushion is also a type of memory foam leads us to our next point—how and when did we invent this strange material? Believe me, this is where, as they say, the plot thickens. Aeronautical engineer Charles Yost helped build a recovery system for the Apollo command module in the year 1962. His work on the Apollo program would lend him another contract with NASA only four years later. And, his time, his job was to assist in the improvement of airline seating for crash and vibration protection, and the development of energy-absorbing techniques for increased survivability. Thus, he created an open-cell, polymeric “memory” foam material with unusual viscoelastic properties. NASA's Ames Research Center fit this material into a new airplane seat design that not only offered better impact protection in the event of an accident, but it also improved passenger comfort—especially on long flights—due to an even distribution of body weight and pressure over the entire contact area.

NASA released memory foam to the public domain in the early 1980s, but even before that, the new tech found its use in medical equipment such as X-ray table pads, and sports equipment such as American football helmet liners. Since then, its technology advanced further—as technology often does—and got cheaper to manufacture, allowing term pain or postural problems. In 2006, a new generation of memory foam was introduced—gel visco or gel memory foam. It consists of gel particles fused with visco foam to reduce trapped body heat, speed up spring back time and help the mattress feel softer”. “As we've discussed in a previous article, pressure sores can and do happen to anyone, but they usually tend to affect people who are on chronic bedrest or who sit in a chair or wheelchair for extended for widespread use. Nowadays, it is commonly found in mattresses, pillows, shoes and blankets. More importantly for us, however, are its medical uses, such as wheelchair memory foam cushions, hospital bed pillows and padding for people suffering long-periods of time.

Pressure relief cushions are designed to reduce pressure on skin by spreading weight over a larger surface area, and they often also minimize friction forces. These cushions must also be breathable in order to reduce any sweat or moisture build-up, which can further worsen pressure sore risk. It's obvious, therefore, that memory foam exhibits most of, if not all, properties desirable for pressure relief.” (Passionate people blog 4-13-21)

A gluteal post-op patient should not wake up after surgery and be lying directly on one's buttocks. Using high density memory foam is proven to prevent pressure, provide cushion to a patient's body and furthermore take the shape of the patient conforming to the patient's body shape. One example of prior art is a hole in an air mattress called a Booty Bean Bag. This product has no head rest no footrest and no memory foam to confirm to the shape of one's body. The Booty Beanbag has reviews stating that a person buttock falls right to the floor and regularly the patient must refill the inflatable plastic, and on www.realself.com a user D, clutch a user of the booty beanbag states “The booty bean bag is, ok I don't know if I overstuffed or what but I cannot get comfortable on it whatsoever, I keep using pillows and blanket to try and make it comfortable, it's impossible to sleep on it since ur not lying all the way back or sitting up. It's been a love hate situation since I been home from surgery” (D Clutch, real self blog May 5, 2021). Another review on www.realself.com states “I bought this and just had surgery June 25. It seems to work for me, but u can't use it for a long period of time. I wish it were made out of a plush material though. I had a BA and BBL and use something else for my sleeping arrangements.” (Eazlove, realself blog Jul. 4, 2018). The Booty bean bag is not equipped to use on a traditional bed it is used on the floor. There are wedge and seat products on the market made of memory foam proven to relieve pressure only while sitting the seat and wedge products provide comfort to one area of the body but not the total body being head, neck, body, buttocks and feet nor provide a negative pressure site for the buttocks. Sleeping on a wedge support device position the buttocks touches and rests on the bed not the wedge device providing absolutely no support to buttocks, a patient also cannot lay flat. Science has proven less pressure is applied to the body is when the body is lying in a flat position. Post BBL or any gluteal surgery a patient's whole body is in pain, black, blue and bruised and there are currently no bed products or device to support the total body providing comfort all in one device postop gluteal surgery allowing a patient to lay or rest in a flat position simultaneously relieving pressure from the buttocks. “Sitting should be limited post buttocks surgery or any buttocks procedure furthermore to 60 minutes in a day, 3× a day.” Wounds-uk.com pg. 32. Science has proven that pressure is medically proven reduced when in a lying position during buttocks surgery recovery to the buttocks so a patient should throughout the day recover lying flat vs sitting. Lying flat vs. sitting applies less pressure on the buttocks in general and is a better method than recovering on a sitting device, A sitting device is although necessary when sitting is necessary.

On web site https://thomasloebmd.com/body/how-to-sleep-after-a-tummy-tuck-brazilian-butt-lift/Ava Lawson states, “After a Brazilian butt lift, it is important to avoid putting pressure where the newly injected fat has been placed to ensure optimum results. Therefore, most patients will be asked to sleep on their stomach or side for at least 4-6 weeks.” (Ava Lawson, Oct. 5, 2019) A patient must not apply pressure to the buttocks before the new fat can stabilize and harvest inside the new buttock augmentation doing so destroys the fat cells and distorts the patients results. Doctors have been advising patients to sleep on stomach as recovery method post buttocks surgery. I have personal experience recovering from this procedure and recovering on the front side is just as painful as the backside without cushion when sleeping or lying down. Wedge pillows are not flat, and they do not support the buttocks nor provide a flat way of sleeping. A patient will spend most of their time in bed for months recovering and they should be comfortable. Medical Grade high density cushion memory foam is used in many medical cushion devices providing comfort to a patient post-op surgery. There does not currently exit a post recovery bed cushion supporting the head body feet and providing a negative pressure therapy site to the buttocks area all in one, keeping the patient in a flat position. A patient will be bed confined for a short period where it is impossible to move immediately after surgery. There is also a time period for up to 6 months before a patient is deemed fully recovered following a BBL procedure or any other gluteal procedure. Mattresses contain springs and are heavy duty; they are neither portable nor lightweight to be travel compatible. Mattress supports do not confirm to one's body. There is a great need for a device to make recovery after a gluteal surgery feasible and comfortable that will offer total body cushioning as well as a negative gluteal pressure site. The present invention a postoperative buttocks recovery bed will provide comfort as prior art US Patient number 9717342 provides sitting comfort to a post off buttocks patient. A medical support device for sitting on has shown scientific proof that memory foam cushions for post-surgery prevent pressure. Only issue is that the prior art U.S. Pat. No. 9,717,342 is for sitting only, there are no prior art devices found in the field configured of memory foam designed to relieve pressure on buttocks for a patient lying flat or sleeping. An inflatable plastic mattress will not maintain firmness it will require to be refilled of air periodically and cannot fit on top of a traditional bed. Air and Plastic will not confirm to a patient's body shape, and air will not conform to a patient's body shape as would memory foam being high density. Following post-op gluteal surgery many women are uncomfortable sleeping on their front side.

None of the prior art methods have been found to be completely suitable to meet these needs and are cumbersome. The present invention provides such a method, and the overall combination of these features is nowhere disclosed in the prior art cited above which appears to be representative of the general art in this area although it is not intended to be an all-inclusive listing of pertinent prior art patents.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, and abstract.

The present invention is a Postoperative Buttocks Recovery Bed which is a recovery bed cushion device head to toe cushion support of high-density polyurethane memory medical grade foam consisting of a negative pressure site. This foam allows a person's whole body to be supported while conforming to the shape of the patient's body, simultaneously providing elevated support for head, neck, knees legs and feet. The present invention is proven to support a person's body relieving pressure on the whole-body head to toe when a patient is resting on the bed device. Current science shows how memory foam is medically proven to mold and support pressure points when used to manufacture medical cushion devices for the human body.

According still another embodiment, the present invention is a method of treating a tissue site is has a hole in the body of the bed where the buttocks will lay to recover, having no pressure applied to the buttocks while in flat laying position. In one alternative embodiment the circular hole is a negative pressure site which is designed for the buttocks since this bed device is a buttocks recovery bed. A The hole will support the healing buttocks by not applying any pressure on the buttocks as the patient recovers from the BBL (Brazilian butt lift) or any other coccyx, gluteal surgery. It is mandatory by doctors when recovering from these medical procedures to recover without applying any pressure to the surgery site. Most patients following this gluteal surgery have just had fat or implants injected into their buttocks. It is medically proven that if you apply any pressure to the buttocks site post op surgery the patient can destroy the operation results. A patient will not be satisfied if they fail to protect the new fat or implant harvested area by not applying any pressure to it for the 2-6-month recovery process. When using postoperative buttocks bed the patient will lie on their backside while the buttocks rests in the hole, the head, neck, knees and feet are supported and elevated. When using the present invention, the patient is in a comfortable traditional sleeping posting lying flat applying no pressure to the buttock.

There currently is no device to support a patient's body while laying on their back to recover post op gluteal surgery that is bed equip able lightweight and recommended by plastic surgeons, patients are instructed to sleep and or lay on their sides or stomach; this present invention is intended to change that old practice. The present invention is intended to allow change to the medical recovery process for BBL aftercare procedure, and or any gluteal surgical procedure. The present invention's purpose is to serve as a total body cushioning device for postoperative recovery following any buttocks surgical procedure. This device will solve the current problem where a patient cannot lay on their back comfortable to recover in comfort without destroying the fat cells and causing tremendous pain to the patient and or destroying the augmentation. The current invention will serve as a body protectant cast during recovery; patients now can sleep on their back while negative therapy pressure serves the buttocks area. While lying on the Postoperative buttock's recovery bed a patient buttocks never comes into direct pressure from the bed. The postoperative buttocks bed will allow an alternative for a BBL or any gluteal surgical patient recovering to do so in ease wherein a patient will be able to lay on the bed device and not apply pressure to buttocks.

It is an object of the invention to provide an improved elements and arrangements thereof in an apparatus for the purposes described which is inexpensive, dependable and fully effective in accomplishing its intended purposes.

This Summary is provided merely for purposes of summarizing some example embodiments, to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 100 is a full view of the invention.

FIG. 200 is a bottom view of the invention.

FIG. 300 is a side view of the bed device.

FIG. 400 is a top view of the invention.

FIG. 500 is bottom view of the bed device.

FIG. 600 is a headrest view of the invention.

FIG. 700 is a footrest view of the invention.

FIG. 800 shows the present invention which comes with a cup holder accessory.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes described in the following specification, are simply exemplary embodiments of the inventive concepts defined in the appended claims. Specific dimensions and other physical characteristics relating to the embodiments disclosed herein are therefore not to be considered as limiting unless the claims expressly state otherwise.

Unless otherwise defined, all terms (including technical and scientific terms) used herein have the same meaning as commonly understood by one having an ordinary skill in the art to which the invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that several techniques are disclosed. Each of these has individual benefits and each can also be used in conjunction with one or more, or in some cases all, of the other disclosed techniques. Accordingly, for the sake of clarity, this description will refrain from repeating possible combination in an unnecessary fashion. Nevertheless, the specifications and claim/s should be read with the understanding that such combinations are entirely within the scope of the invention and the claim/s.

The method of the present invention is that it relates to a postoperative buttocks bed for recovery, a high-density medical grade foam bed comprised of a head, body, and footrest with a negative pressure site, the hole in the body of the bed. The current invention brings a device for recovery that will allow a patient to recover on their backside lying flat post-op any gluteal procedure while providing an all-in-one total body support device. Made of medical grade a support device is proven to remove pressure and provide comfort during any postop recovery. The current invention will protect the implant or the newly harvested fat the patient has just received by allowing the buttocks to rest in an airspace hole offering negative pressure therapy. It is important not to apply pressure to the gluteal site post-op because a patient's new fat or implant will not harvest. When a patient rests or sleeps which is all a patient can do post-op, the present invention provides comfort minimizing the pain experienced during the recovery process. The buttocks are so bruised and soar that when lying on ones back the buttocks rest softly inside a hole with a cushion backside bottom. The buttocks rest in the air space having a bottom cushion backing to allow the patient to never fall through support device and touch the floor. A postoperative buttocks recovery bed provides support to all painful areas of the body post any gluteal surgery such as a BBL.

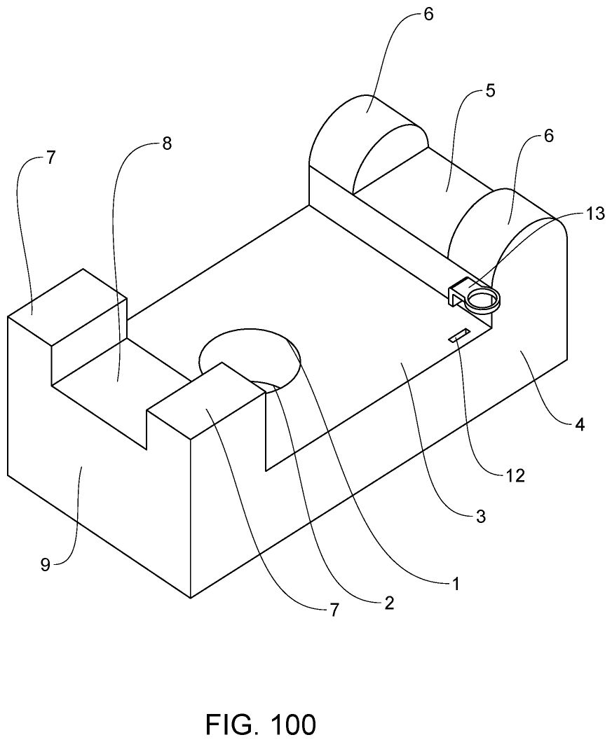

If we see the FIG. 100 the hole 1 can range from any size diameter stand sizes being 7″ hole to 24″ hole, depending on the size of bed or patient's size request. The hole 1, is the negative pressure site to provide the airspace where the buttocks will rest while the patient is lying flat on the patient's backside. The hole 1 has a backside of hole 2 for providing a cushion between the patients buttocks and the surface the present invention lays, on such as a bed or floor. The body of the bed 3 supports all a patient's weight when a patient is lying on one's backside. The middle of the body 3 is connected at one end with an elevated built-in pillow headrest 5 and a foot, knee, and leg rest 8. The foot, knee, and leg rest 8 have side panels 7 which create a wall providing comfort and does not allow a patient's bottom leg portion to fall off the bed. The Headrest 5 supports the neck and head has side panels 6 that also support the side of the neck and head, keeping them in position from falling off the bed.

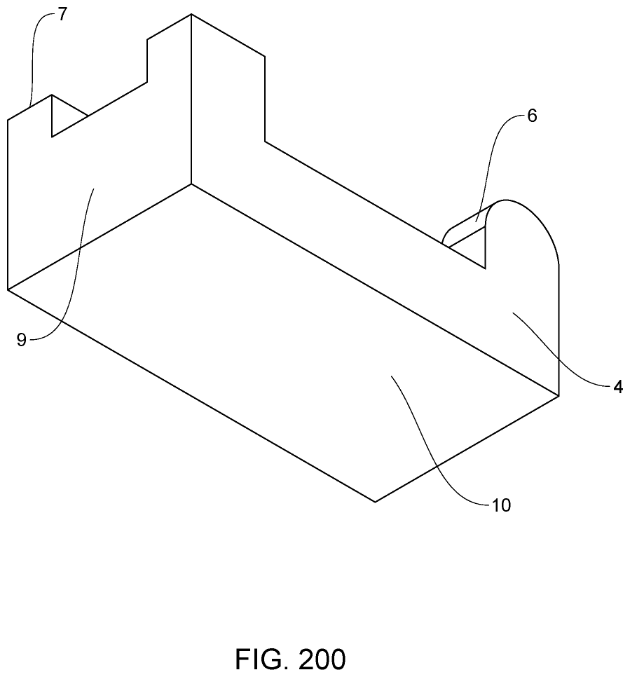

If we see FIG. 200 5 is the head rest designed to support a patients head as the patient lays in the bed device, being a built In pillow to the bed device, connected with side head panels 6 prevents the patient's body from rolling or falling of the bed device. The bottom of the bed 10 has a high density medical grade compression never allowing a patient to fall or sink to the floor. 7 are the side panel views to knee, leg, and foot support creating walls to hold the patient inside the bed. 7 are views side panel leg, foot, and knee rest attached to 9 the back panel of the devices foot, knee, and leg rest, designed to provide total body support to the patient when a patient sleeps or rest in the bed device.

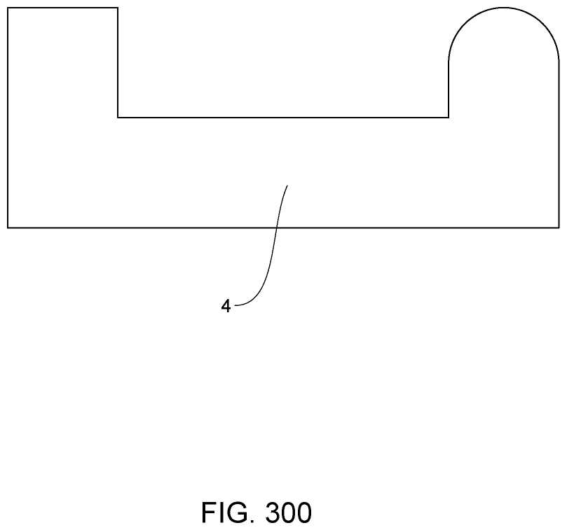

In FIG. 300 it shows the side view of the present inventions 4 side panel of bed device.

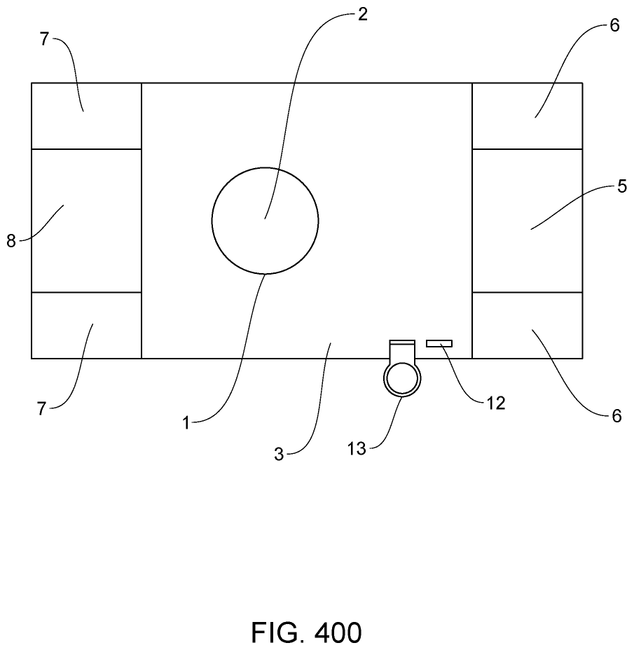

In FIG. 400 top view of 1 the hole is a negative pressure site where the buttocks rest while in laying position. 5 is the head rest attached to 6 head rest side panels supporting and elevating a patient's head and neck. 7 is the side panel foot, knee, and leg rest providing a wall barrier side panel to 8 the actual foot knee and leg elevated support. 2 shows the backside to the negative pressure site providing comfort and a barrier underneath the buttocks. 12 is an insert slit to show where the accessory 13 the cup holder needs to be placed by patient to add the accessory to the bed upon purchase.

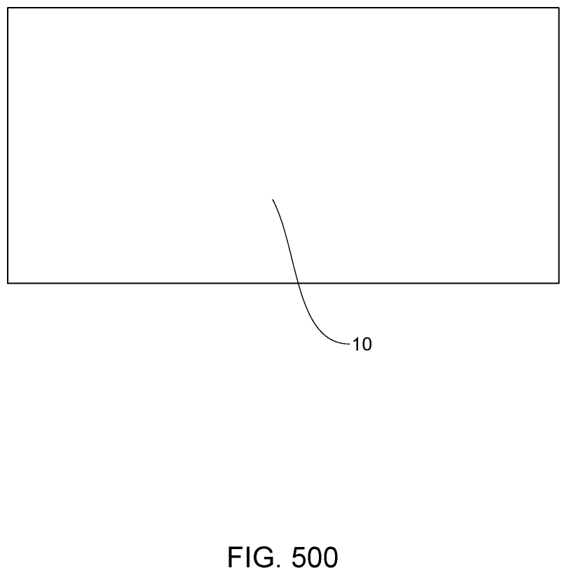

FIG. 500 is the bottom view 10 of the present invention capable of going onto a traditional bed or a floor flat surface.

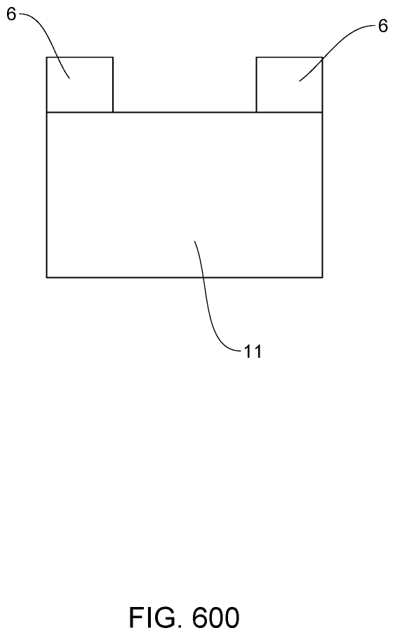

FIG. 600 is the head rest view of the present invention, and 11 being the back support panel to head rest and 6 being the head rest's side panels configured to create the shape of a bed.

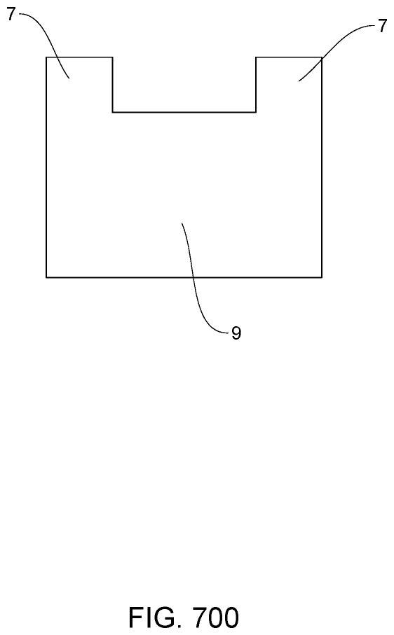

FIG. 700 is the footrest view of the present invention, and 9 being the back panel support to the knee, leg, and footrest and 7 being the side panels configured to create the shape of a bed.

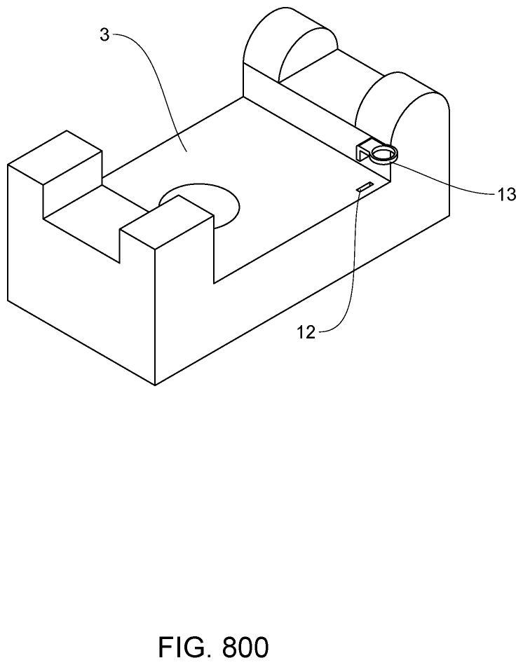

If we see FIG. 800 the present invention comes with a cup holder accessory 13, 12 is the slit on top of 3 the body of the bed. 13 is the actual cup holder that has will be inserted into 12 the slit on 3 the body of the bed as an attachable accessory. The cup holder 13 is useful for daily drinking fluids that the patient will need for taking medicine. This accessory 13 the cup holder will help a bedridden patient from depending on others to hand them beverages every so often. Once a patient receives the present invention, they must insert the cupholder 13, hence 13 is an accessory only and not apart of the present bed device's manufacturing process.

The postoperative buttock's recovery bed is equipped with a cup holder to provide the patient a convenient way to access fluids. A patient must stay hydrated while recovering for a 6-8-week mandatory bed rest period. Drinking fluids are necessary at arm's reach enabling a convenient way for a patient to take medicines for pain and antibiotics which are required post any surgical procedure. A current problem exists when a patient is in pain and must ask a nurse or assistant to access drinking fluids from a nightstand or table. The nightstand or table is more than likely not at arm's reach of the patient making it even harder for a patient to recover.

The present invention being a total body cushion support device intended to cast a patient's body relieving the gluteal area of any touch, pain, or pressure and total pressure support to the body. The neck is supported and elevated and are the feet; the invention is a total bed body support device assuring no pressure to the gluteal area of the patient, all in one device postop buttocks recover bed. The present invention is manufactured in high density medical grade foam. This foam conforms to the patient's body and is certified high density foam to provide the best support for a patient's comfort; the pressure site can change per custom order per patient's body size in diameter. The present invention is lastly dipped in medical grade liquid vinyl during the manufacturing process. The liquid vinyl dries to take form of the high-density foam bed. The vinyl now coats the foam being a medical grade vinyl approved for medical devices that can clean the pad. The liquid vinyl protects the foam and provides a skin barrier between the patient and the foam. The skin barrier medical grade vinyl used is wipeable for cleaning and soft against the patient's skin.

Referring to the drawing, FIG. 100-800 the captions refer to:

1. Hole;

2. Base of the hole negative pressure site.

3. Body.

4. Side Panel.

5. Head rest elevated pillow.

6. Side panels to head rest

7. Side panels and support of the bed for knees legs and feet providing elevation.

8. knee, legs and footrest.

9. Back panel to foot knee and leg rest.

10. Bottom of bed device.

11. Back panel support to headrest.

12. Slit inside of body of bed device for cupholder insert

13. The cupholder accessory

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A cushion device for enabling a patient to recover postoperative a BBL surgery or any other Buttocks Augmentation surgical procedure on their back Laying flat and or sleeping while comprising:
a hole with a backside barrier any shape and size embedded in the body middle portion of the bed creating a negative pressure site where the buttock lays comprising:
a top region elevated built in pillow head rest allowing a patient's neck and head to rest and be elevated like a traditional bed further comprising;
a lower region built in knee foot and leg rest that can be any length providing total body support.

2. The bed device of claim 1 further comprising manufacturing of 100 percent of foam any type of foam any type being medical grade, being medical grade scientifically supports a patient's total body and relieve pressure from the buttocks.

3. The bed device of claim 2 further comprising of an outer medical grade vinyl dipping coating wipeable for cleaning becoming a medical skin membrane to the foam wherein a patient's skin is comfortable, and doctor approved to directly touch the bed device.

4. The foam is said in claim 3 not to be limited to the only covering a bed device in claim 3 may have, an example would be the bed device comprising of a fabric covering or no covering at all on the bed device.

5. A bed device of claim 4 further comprising a cupholder accessory allowing convenience for a patient to access a cup or drink at arm's reach while in a laying position.

6. A bed device in claim 5 further compromising of a device that can be carried by a patient during travel and is equip able for the floor or the bed.

7) A method allowing a patient after any Buttocks surgical procedure to recover lying flat on the backside simultaneously applying no pressure to the buttocks.
